(19) United States
(12) Patent Application Publication
(10) Pub. No.: US 2025/0381126 A1
(43) Pub. Date: Dec. 18, 2025

(54) POLYVINYL ALCOHOL FILM-FORMING POLYMERS FOR ALCOHOL-BASED SUNSCREEN FORMULATIONS AND METHODS OF USING SAME

(71) Applicant: NOURYON CHEMICALS INTERNATIONAL B.V., Amsterdam (NL)

(72) Inventors: Hanamanthsa Shankarsa BEVINAKATTI, Somerset, NJ (US); John Socrates THOMAIDES, Berkeley Heights, NJ (US); Qiwei HE, Belle Mead, NJ (US)

(73) Assignee: NOURYON CHEMICALS INTERNATIONAL B.V., Amsterdam (NL)

(21) Appl. No.: 18/878,357

(22) PCT Filed: Jun. 26, 2023
(86) PCT No.: PCT/EP2023/067253

§ 371 Date: Dec. 23, 2024

Related U.S. Application Data
(60) Provisional application No. 63/355,177, filed on Jun. 24, 2022.

(51) Int. Cl. A61K 8/81; A61K 8/31; A61K 8/34; A61Q 17/04

(52) U.S. Cl. CPC A61K 8/8135; A61K 8/315; A61K 8/34; A61Q 17/04; A61K 2800/31; A61K 2800/87; A61K 2800/95

(57) ABSTRACT

The present disclosure relates to a sunscreen formulation comprising: (a) a volatile solvent; and (b) at least one film-forming polymer comprising at least one of: (i) polyvinyl alcohol or (ii) polyvinyl alcohol ester said polyvinyl alcohol ester having ester functionality other than solely acetate, and (i) and (ii) being soluble in the volatile solvent, provided that if the at least one film-forming polymer comprises polyvinyl alcohol, then the polyvinyl alcohol has a degree of hydrolysis less than about 60%. Methods of using the sunscreen formulations to protect skin against the damaging effects of sun are also disclosed.

### CROSS-REFERENCE TO RELATED APPLICATIONS

This application is a U.S. National-Stage entry under 35 U.S.C. §371 based on International Application No. PCT/EP2023/067253, filed Jun. 26, 2023, which was published under PCT Article 21(2) and which claims priority of U.S. Provisional Patent Application No. 63/355,177, filed Jun. 24, 2022, the entire contents of which patent application are hereby incorporated herein by reference.

### FIELD OF THE DISCLOSURE

The present disclosure relates to film-forming polymers for sunscreen formulations and to methods of using the sunscreen formulations.

### BACKGROUND

Polymers used in sunscreen applications have conventionally been made using synthetic materials. In order for the polymers to be suitable in such sunscreen applications, they should be soluble in alcohol-based systems and/or systems containing low amounts of volatile organic compounds (VOC), and in the case of aerosol-based sunscreen sprays, they should also be compatible with the propellant. Conventional synthetic polymers are generally inexpensive and provide acceptable performance. However, because they are not biodegradable, conventional synthetic polymers are not sustainable. In addition, replicating the cost and performance of synthetic polymers is not easy.

Accordingly, there is a need for biodegradable sunscreen polymers that provide equal to or better performance at comparable costs than the non-biodegradable alternatives and that are soluble in alcohol-based systems, such as ethanol-based systems and optionally, that are compatible with propellants, such as hydrocarbons or dimethyl ether.

Suncare formulations based on biodegradable polyesters are the subject of our recent publications: US 2021/0259930; US 2021/0259945; and US 2021/0259946.

U.S. Pat. No. 6,602,489 and WO 2006/018328 describe compact hair spray products comprising a film-forming polymer, which could be a polyvinyl alcohol sold by Kuraray under the tradename Poval™. The available Poval™ products have DH>99%. Kuraray usually provides these materials at certain viscosities without any indication of molecular weight. For example, Poval™ LM-10 HD is supplied at a viscosity of 4.5-5.7 MPa·s. Poval™ LM-20 is supplied at a viscosity of 3.0-4.0 MPa·s. Interestingly, however, Example 1 of U.S. Pat. No. 7,939,582 provides the number average molecular weight of Poval™ LM-20 as about 20,000, but it is not known if this is accurate. The polyvinyl alcohol could also be those available from Sekisui, for example, those products available under the tradenames Selvol™ PVOH 523/E 523 and Selvol™ PVOH 540, both of which have a DH of 88%.

US 2015/0328130 describes a sunscreen composition comprising 0.1 to 10% of a non-linear polymer selected from a group that includes polyvinyl alcohol; and 40 to 80% water. The exemplified compositions include various polyvinyl alcohols having degrees of hydrolysis ranging from 86-99%; and at least 70% water; but do not comprise ethanol.

AU 2017/204073 describes cosmetic compositions, in particular, sunscreens, spiked with polyvinyl alcohol to facilitate washing UV filters out of textiles contaminated with these compositions. According to the teachings, the addition of polyvinyl alcohol to the compositions has an advantageous effect on washability, reducing textile staining caused by the compositions. The effect is demonstrated with polyvinyl alcohol having a degree of hydrolysis of 86 to 89 mol %.

U.S. Pat. No. 3,417,180 describes hair spray formulations comprising polyvinyl acetates partially hydrolyzed to the extent of about 15 to 60% of their acetate groups. Solubility up to 50% is demonstrated in anhydrous ethanol and up to 48% in halocarbon propellant, particularly trichlorofluoromethane and dichlorodifluoromethane. The use of hydrocarbon propellants and ethers is neither taught nor suggested, nor is the solubility of the partially hydrolyzed polyvinyl acetates in such propellants demonstrated. Copolymers of partially hydrolyzed polyvinyl acetate and other monomers, including (meth)acrylic acid and crotonic acid are theorized but not exemplified.

U.S. Pat. No. 3,005,809 describes sheets or films of vinyl alcohol-crotonic acid copolymers.

The compatibility of film-forming polymers with alcohol-based, especially ethanol-based sunscreen formulations, remains a significant problem for the formulator and there is a significant need for film-forming polymers that are biodegradable yet at the same time are soluble in and compatible with alcohol-based systems.

Accordingly, it is an object of the present disclosure to provide film-forming polymers that are biodegradable, exhibit excellent compatibility with alcohol-based formulations, especially ethanol-based formulations, and, therefore, can be formulated into stable sunscreen formulations that provide biodegradility. In addition, other objects, desirable features and characteristics will become apparent from the subsequent summary and detailed description, and the appended claims, taken in conjunction with the accompanying drawings and this background.

### BRIEF SUMMARY

The present disclosure relates in one embodiment to a sunscreen formulation comprising:

(a) a volatile solvent; and
(b) at least one film-forming polymer comprising at least one of: (i) polyvinyl alcohol or (ii) polyvinyl alcohol ester, said polyvinyl alcohol ester having ester functionality other than solely acetate, and (i) and (ii) being soluble in the volatile solvent; and
(c) at least one sunscreen active agent.

The present disclosure relates in a second embodiment to a method of protecting a user to be exposed or already exposed to sunlight from the damaging effects of exposure to sunlight, the method comprising applying to skin of said user an effective amount therefor of the sunscreen formulation described herein.

### DETAILED DESCRIPTION

The following detailed description is merely exemplary in nature and is not intended to limit the present disclosure or the application and uses of the present disclosure. Furthermore, there is no intention to be bound by any theory presented in the preceding background of the present disclosure or the following detailed description. It is to be appreciated that all numerical values as provided herein, save for the actual examples, are approximate values with endpoints or particular values intended to be read as “about” or “approximately” the value as recited.

Polyvinyl alcohol (“PVOH”) has the generalized structure:

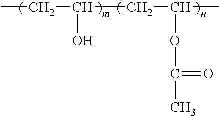

and is characterized by pendent OH and acetate (CH₃—CO—) groups/functionality.

There are several methods known for the manufacturing of PVOH. However, from an industrial point of view, the current mainstream method of production is as follows: Polyvinyl acetate is manufactured by radical polymerization using vinyl acetate as a raw material and then the polyvinyl acetate is hydrolyzed/transesterified. Methanol or ethanol are mainly used as a solvent in the polymerization process/hydrolysis process.

The basic properties of PVOH are typically governed by the degree of polymerization and the degree of hydrolysis.

The degree of polymerization of PVOH is generally indicated by the viscosity-average degree of polymerization derived from the viscosity in water. Additionally, when expressing the degree of hydrolysis using the repeating units m and n, the degree of hydrolysis can be expressed as follows: Degree of hydrolysis (mol %)=m/(n+m)×100. This is the value with which the proportion of the vinyl alcohol unit in all the repeating units is indicated in mol %.

We have discovered that the degree of hydrolysis is an important parameter with respect to the solubility of the base polyvinyl alcohol in the volatile solvent. On the other hand, when the base polyvinyl alcohol is derivatized with hydrophobic functional groups, the degree of hydrolysis is not as important.

In one embodiment, the film-forming polymer comprises underivatized polyvinyl alcohol (referred to herein alternatively as “base polyvinyl alcohol”).

In a preferred embodiment, the base polyvinyl alcohol has a degree of hydrolysis of 60% or less, especially 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and/or 60%; or a continuous sequence of percentages within this range, for example, 1% to 60%, 1% to 59%, 2% to 59%, 2% to 58%, 3% to 58%, etc.

In another preferred embodiment, the base polyvinyl alcohol has a degree of hydrolysis of 20% to 58%, especially 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, and/or 58%; or a continuous sequence of percentages within this range, for example, 20% to 58%, 21% to 58%, 21% to 57%, 22% to 57%, 22% to 56%, etc.

In another preferred embodiment, the base polyvinyl alcohol has a degree of hydrolysis of 35% to 58%, especially 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, and/or 58%; or a continuous sequence of percentages within this range, for example, 35% to 58%, 36% to 58%, 36% to 57%, 37% to 57%, 37% to 56%, etc.

In the case of a range qualified with the term “about,” for example, “less than about 60%” or “about 35% to about 58%,” the range is to be expanded a maximum of 1%, preferably at most 0.5%.

Once the base PVOH has been prepared, the base PVOH can be esterified by further reaction with a suitable functionalizing agent, for example, an acid, an anhydride, or a lactone, according to methods known in the art. It is also possible to prepare the ester starting with vinyl acetate and other vinyl monomers at specified ratios to form an intermediate copolymer having pendent acetate functionality and then hydrolyzing the acetate group selectively to give desired PVOH portion.

Thus, in another embodiment, the at least one film-forming polymer comprises polyvinyl alcohol derivatized with additional ester functionality different from the pendent OH and acetate groups that characterize the base polyvinyl alcohol.

As used herein, simply “polyvinyl alcohol” or “PVOH” without more, or “base polyvinyl alcohol” or “base PVOH” means polyvinyl alcohol including only pendent OH and acetate groups.

As used herein, “polyvinyl alcohol esters” or “PVOH esters” or the like means polyvinyl alcohol derivatized with additional ester functionality different from the pendent OH and acetate groups that characterize the base polyvinyl alcohol.

As noted above, one way PVOH esters can be formed is by reacting the base PVOH with a suitable acid or a suitable acid derivative, such as an anhydride or lactone.

Suitable acids include linear, branched or cyclic, saturated or unsaturated, nonaromatic or aromatic monocarboxylic acids or polycarboxylic acids.

Exemplary acids useful for this purpose include, without being limiting, propionic acid, butyric acid, valeric acid, isovaleric acid, caproic acid, heptanoic acid, octanoic acid, nonanoic acid, decanoic acid, dodecanoic acid, octadecanoic acid, pivalic acid, 2-ethylhexanoic acid, isononanoic acid, neononanoic acid, neodecanoic acid, Versatic Acid 9 (highly branched nonanoic acid available from Hexion), Versatic Acid 10 (highly branched decanoic acid available from Hexion), palmitic acid, benzoic acid, anthranilic acid, salicylic acid, phenyl acetic acid, cinnamic acid, 4-chlorobenzoic acid, 1,4-dichlorobenzoic acid, 4-nitrobenzoic acid, 2,4-dinitrobenzoic acid, succinic acid, glutaric acid, adipic acid, octanedioic acid (suberic acid), malonic acid, malic acid, octenyl succinic acid, dodecenyl succinic acid, azelaic acid, sebacic acid, pimelic acid, 2,2-dimethylsuccinic acid, 3,3-dimethylglutaric acid, 2,2-dimethylglutaric acid, maleic acid, fumaric acid and itaconic acid-enanthic acid, n-caprylic acid, n-pelargonic acid, n-capric acid, n-lauric acid, n-myristic acid, n-stearic acid, oleic acid, oxalic acid, malonic acid, pimelic acid, azelaic acid, sebacic acid, benzoic acid, phthalic acid, citric acid, tartaric acid, lactic acid, malic acid, toluic acid, isophthalic acid, phthalic acid, terephthalic acid, trimellitic acid, 1,2-cyclohexane dicarboxylic acid, 1,3-cyclohexane dicarboxylic acid, tetrahydrophthalic acid, endomethylene tetrahydrophthalic acid, hexahydrophthalic acid, tetrachlorophthalic acid, methyl hexahydrophthalic acid, substituted derivatives thereof, as well as mixtures of one or more of the foregoing.

Anhydrides of the above acids, where they exist, can also be employed, such as succinic anhydride, dimethyl ester and diethyl ester of malonic acid, octenyl succinic anhydride, dodecenyl succinic anhydride, phthalic anhydride, trimellitic anhydride, tetrahydrophthalic anhydride, endomethylene tetrahydrophthalic anhydride, hexahydrophthalic anhydride, methyl hexahydrophthalic anhydride.

In addition, certain materials that react in a manner similar to acids to form esters are also useful. Such materials include lactones, including caprolactone, propylolactone, methyl caprolactone, butyrolactone.

As also noted above, PVOH esters can be made by free-radical polymerization of vinyl acetate and other vinyl monomers at specified ratios to form an intermediate copolymer having pendent acetate functionality and then hydrolyzing the acetate group selectively to give desired PVOH portion. Polymerization methods known in the art such as but not limited to solution polymerization, bulk polymerization, precipitation polymerization, emulsion polymerization, and suspension polymerization can be used to polymerize vinyl acetate with other vinyl monomers. In one preferred embodiment, the polymerization is a solution polymerization. In another preferred embodiment, the polymerization is a suspension polymerization.

The disclosed sunscreen formulations additionally comprise a volatile solvent. Examples of volatile solvents include one or more of alcohols, such as methanol, ethanol and isopropanol; volatile hydrocarbons, such as isooctane, isododecane, and isohexadecane; volatile aldehydes; volatile silicones; and volatile ketones, such as acetone and methyl ethyl ketone. Hydrofluoro-olefins may also be used as a carrier solvent in the formulations.

In a preferred embodiment, the volatile solvent is an alcohol-based solvent system, wherein the alcohol-based solvent system comprises at least one C₁₋₆ straight or branched chain alcohol.

In another preferred embodiment, the volatile solvent is a ketone.

In an especially preferred embodiment, the volatile solvent is chosen from the group consisting of ethanol, methanol, isopropanol, acetone, and mixtures thereof.

In another especially preferred embodiment, the sunscreen formulations comprise ethanol.

In a more preferred embodiment, the sunscreen formulations comprising ethanol are anhydrous. The terms “non-aqueous” and “anhydrous” are used interchangeably herein and refer to compositions containing less than about 10% by weight water, especially less than about 5% by weight water, or less than 1% by weight water, or even 0% water.

In a most preferred embodiment, the sunscreen formulation comprises ethanol but not any other alcohol or water.

In an alternative embodiment, where water is present, this small amount of water may be desirable, for example as a processing aid or co-solvent. In certain example embodiments, the water contents of the compositions will be no greater than about 9% water so as to prevent the active to phase-separate or precipitate out of solution. Those of ordinary skill in the art will recognize that different actives have different tolerances for water in solution and will adjust water content accordingly.

In an embodiment, the polymer is fully soluble in an ethanol or a predominately ethanol mixture with 0-20 wt %, preferably 5-10% water.

In another embodiment, the polymer is fully soluble in ethanol.

In another embodiment, the polymer is fully soluble in anhydrous ethanol.

In another embodiment, the polymer is fully soluble in anhydrous ethanol immediately after the polymer is incorporated into the solvent. The phrase “immediately after” as used throughout this disclosure means an hour or less, preferably a half-hour or less, most preferably 15 minutes or less.

In another embodiment, the polymer is remains fully soluble in anhydrous ethanol after 24 hours after the polymer is incorporated into the solvent.

In an especially preferred embodiment, the polymer is:

(a) fully soluble in anhydrous ethanol immediately after the polymer is incorporated into the solvent; and
(b) fully soluble in anhydrous ethanol after 24 hours after the polymer is incorporated into the solvent.

In an even more preferred embodiment, the polymer comprises polyvinyl alcohol or an ester thereof, wherein if the polymer comprises underivatized polyvinyl alcohol, the polyvinyl alcohol has a degree of hydrolysis of 35-58% percent and is:

(a) fully soluble in anhydrous ethanol immediately after the polymer is incorporated into the solvent; and
(b) fully soluble in anhydrous ethanol after 24 hours after the polymer is incorporated into the solvent.

In a more preferred embodiment, the polymer comprises polyvinyl alcohol or an ester thereof, wherein if the polymer comprises underivatized polyvinyl alcohol, the polyvinyl alcohol has a degree of hydrolysis of 35-45% and is:

(a) fully soluble in anhydrous ethanol immediately after the polymer is incorporated into the solvent; and
(b) fully soluble in anhydrous ethanol after 24 hours after the polymer is incorporated into the solvent.

In an even more preferred embodiment, the polymer comprises polyvinyl alcohol or an ester thereof, wherein if the polymer comprises underivatized polyvinyl alcohol, the polyvinyl alcohol has a degree of hydrolysis of 38-42% percent and is:

(a) fully soluble in anhydrous ethanol immediately after the polymer is incorporated into the solvent; and
(b) fully soluble in anhydrous ethanol after 24 hours after the polymer is incorporated into the solvent.

In one embodiment, the sunscreen formulations further comprise a propellant.

In certain embodiments of the subject disclosure, the disclosed formulations can be stored in containers under pressure by combination with the propellant and the disclosure extends to such containers under pressure containing the disclosed formulations. The formulations thus stored can be applied by opening a valve in the container releasing the propellant and the composition, typically in a spray or mist. The propellant used in the composition may be any suitable gas, or combination of gasses, that can be compressed or liquefied within a dispensing spray canister, which expand or volatilize to vapor or gas form upon exposure to ambient temperature and pressure conditions to deliver the composition in an aerosol form. Suitable propellants include hydrocarbons having 1 to 5 carbon atoms, including but not limited to methane, ethane, propane, isopropane, butane, isobutane, butene, pentane, isopentane, neopentane, and pentene, hydrofluorocarbons (HFCs), including but not limited to 1,1-difluoroethane (HP 152a), chlorofluorocarbons (CFCs), hydrofluoro-olefins (HFOs), nitrogen, ethers including dimethyl ether, and any mixtures thereof. Those of ordinary skill in the art recognize that in a closed container such as an aluminum can or glass bottle, propellants such as dimethyl ether condense to the liquid state at ambient temperature. Thus, the composition in the aerosol container is a liquid formulation that can contain dissolved propellant, undissolved liquid propellant and gaseous propellant. All of this is under pressure due to the vapor pressure of the propellant. In the practice of this aspect of the subject disclosure, the propellant can be present in an amount up to about 90 weight percent, preferably from about 2 weight percent to about 50 weight percent, and more preferably about 5 weight percent to about 40 weight percent, more preferably at about 30 weight percent, based on the total weight of the aerosol composition.

In another embodiment, the propellant is a hydrocarbon-derived ether.

In another embodiment, the propellant is dimethyl ether (DME).

In a preferred embodiment, the polymer is fully compatible with DME.

In an especially preferred embodiment, the polymer is:

(a) fully soluble in anhydrous ethanol immediately after the polymer is incorporated into the solvent;
(b) fully soluble in anhydrous ethanol after 24 hours after the polymer is incorporated into the solvent; and
(c) fully compatible with DME.

In an even more preferred embodiment, the polymer comprises polyvinyl alcohol or an ester thereof, wherein if the polymer comprises underivatized polyvinyl alcohol, the polyvinyl alcohol has a degree of hydrolysis of 35-58% percent and is:

(a) fully soluble in anhydrous ethanol immediately after the polymer is incorporated into the solvent;
(b) fully soluble in anhydrous ethanol after 24 hours after the polymer is incorporated into the solvent; and
(c) fully compatible with DME.

In a more preferred embodiment, the polymer comprises polyvinyl alcohol or an ester thereof, wherein if the polymer comprises underivatized polyvinyl alcohol, the polyvinyl alcohol has a degree of hydrolysis of 35-45% and is:

(a) fully soluble in anhydrous ethanol immediately after the polymer is incorporated into the solvent;
(b) fully soluble in anhydrous ethanol after 24 hours after the polymer is incorporated into the solvent; and
(c) fully compatible with DME.

In an even more preferred embodiment, the polymer comprises polyvinyl alcohol or an ester thereof, wherein if the polymer comprises underivatized polyvinyl alcohol, the polyvinyl alcohol has a degree of hydrolysis of 38-42% percent and is:

(a) fully soluble in anhydrous ethanol immediately after the polymer is incorporated into the solvent;
(b) fully soluble in anhydrous ethanol after 24 hours after the polymer is incorporated into the solvent; and
(c) fully compatible with DME.

In another embodiment, the propellant is a hydrofluorocarbon.

In another embodiment, the propellant is 1,1-difluoroethane.

In a preferred embodiment, the polymer is fully compatible with 1,1-difluoroethane.

In an especially preferred embodiment, the polymer is:

(a) fully soluble in anhydrous ethanol immediately after the polymer is incorporated into the solvent;
(b) fully soluble in anhydrous ethanol after 24 hours after the polymer is incorporated into the solvent; and
(c) fully compatible with 1,1-difluoroethane.

In a more preferred embodiment, the polymer comprises polyvinyl alcohol or an ester thereof, wherein if the polymer comprises underivatized polyvinyl alcohol, the polyvinyl alcohol has a degree of hydrolysis of 35-58% and is:

(a) fully soluble in anhydrous ethanol immediately after the polymer is incorporated into the solvent;
(b) fully soluble in anhydrous ethanol after 24 hours after the polymer is incorporated into the solvent; and
(c) fully compatible with 1,1-difluoroethane.

In a more preferred embodiment, the polymer comprises polyvinyl alcohol or an ester thereof, wherein if the polymer comprises underivatized polyvinyl alcohol, the polyvinyl alcohol has a degree of hydrolysis of 35-45% and is:

(a) fully soluble in anhydrous ethanol immediately after the polymer is incorporated into the solvent;
(b) fully soluble in anhydrous ethanol after 24 hours after the polymer is incorporated into the solvent; and
(c) fully compatible with 1,1-difluoroethane.

In an even more preferred embodiment, the polymer comprises polyvinyl alcohol or an ester thereof, wherein if the polymer comprises underivatized polyvinyl alcohol, the polyvinyl alcohol has a degree of hydrolysis of 38-42% percent and is:

(a) fully soluble in anhydrous ethanol immediately after the polymer is incorporated into the solvent;
(b) fully soluble in anhydrous ethanol after 24 hours after the polymer is incorporated into the solvent; and
(c) fully compatible with 1,1-difluoroethane.

In one embodiment, the sunscreen formulation disclosed here is dispensed from a bag-on-valve device. Generally, bag-on-valve devices comprise a spray can fitted with an aerosol valve and comprising a welded bag. The product is placed inside the bag while the propellant is filled in the space between the bag and the can. The product is dispensed by the propellant simply squeezing the bag when the spray button is pressed.

Generally, the compositions according to the disclosure are prepared as non-aqueous, volatile solvent-based compositions. However, in some embodiments, the compositions comprise a single liquid phase that may further comprise dispersed particulates, for example, UV active agents in particulate form.

We have found that in certain instances the base polyvinyl alcohol polymer as described herein above, which contains predominantly only pendent alcohol and acetate groups, can be derivatized without loss of solubility in the volatile solvent.

The solubility of the derivatized polyvinyl alcohol in such volatile solvent can be assessed by using the testing protocols set forth in the examples hereinbelow.

As noted above, the main production method for producing polyvinyl alcohol involves manufacturing polyvinyl acetate by radical polymerization using vinyl acetate as a raw material and then hydrolyzing the polyvinyl acetate. Derivatives can then be made by, for example, (a) reacting polyvinyl alcohol with an acid, an anhydride, or a lactone; or (b) directly by (i) polymerizing a polymerizable monomer precursor of the derivatizing functional group, for example, a vinyl alkanoate, along with the vinyl acetate followed by (ii) hydrolysis to create alcohol functionality in any desired degree of hydrolysis.

In one embodiment, the polyvinyl alcohol is derivatized with crotonic acid, for example, by copolymerizing crotonic acid with vinyl acetate, the product of which is then hydrolyzed/transesterified to yield the desired polyvinyl alcohol derivative. In a preferred embodiment, the product is a partially hydrolyzed vinyl acetate-crotonic acid copolymer comprising pendant carboxylic acid groups and pendant hydroxy groups condensed together to form an internal gamma-lactone functionality.

In one embodiment, the polyvinyl alcohol polymer is derivatized with succinic acid, for example, by reacting a polyvinyl alcohol soluble in the volatile solvent with succinic anhydride.

In another embodiment, the polyvinyl alcohol polymer is derivatized with caprolactone, for example, by reacting a polyvinyl alcohol soluble in the volatile solvent with caprolactone.

In another embodiment, the polyvinyl alcohol polymer is derivatized with pivalic acid, for example, by copolymerizing vinyl pivalate with vinyl acetate, the product of which is then hydrolyzed/transesterified to yield the desired polyvinyl alcohol derivative.

In another embodiment, the polyvinyl alcohol polymer is derivatized with neononanoic acid, for example, by copolymerizing vinyl neononanoate with vinyl acetate, the product of which is then hydrolyzed/transesterified to yield the desired polyvinyl alcohol derivative.

In another embodiment, the polyvinyl alcohol polymer is derivatized with Versatic acid 9, for example, by copolymerizing the vinyl ester of Versatic acid 9 (e.g., VeoVa 9 from Hexion) with vinyl acetate, the product of which is then hydrolyzed/transesterified to yield the desired polyvinyl alcohol derivative.

In another embodiment, the polyvinyl alcohol polymer is derivatized with neodecanoic acid, for example, by copolymerizing vinyl neodecanoate with vinyl acetate, the product of which is then hydrolyzed/transesterified to yield the desired polyvinyl alcohol derivative.

In another embodiment, the polyvinyl alcohol polymer is derivatized with Versatic Acid 10, for example, by copolymerizing vinyl neodecanoate (e.g., VeoVa 10 from Hexion) with vinyl acetate, the product of which is then hydrolyzed/transesterified to yield the desired polyvinyl alcohol derivative.

In another embodiment, the polyvinyl alcohol polymer is derivatized with 2-ethylhexanoic acid, for example, by polymerizing vinyl 2-ethylhexanoate with vinyl acetate, the product of which is then hydrolyzed/transesterified to yield the desired polyvinyl alcohol derivative.

In another embodiment, the polyvinyl alcohol polymer is derivatized with lauric acid, for example, by polymerizing vinyl laurate with vinyl acetate, the product of which is then hydrolyzed/transesterified to yield the desired polyvinyl alcohol derivative.

In another embodiment, the poly (vinyl alcohol) polymer is derivatized with a carboxylic acid pendent from the polymer backbone yielding an internal lactone (cyclic ester) moiety.

In a preferred embodiment, the carboxylic acid pendent from the polymer backbone reacts with an alcohol pendent from the gamma-carbon relative to the carbon of the pendent carboxylic acid to form an internal gamma-lactone (five-membered ring).

In a preferred embodiment, the carboxylic acid pendent from the polymer backbone is the residue of acrylic acid or crotonic acid obtained after copolymerization of acrylic acid or crotonic acid, respectively, with vinyl acetate and, optionally, one or more vinyl alkanoates followed by hydrolysis to create alcohol functionality in any desired degree of hydrolysis.

In a more preferred embodiment, the carboxylic acid pendent from the polymer backbone is the residue of crotonic acid obtained after copolymerization of crotonic acid with vinyl acetate and, optionally, one or more vinyl alkanoates followed by hydrolysis to create alcohol functionality in any desired degree of hydrolysis.

In a most preferred embodiment, the carboxylic acid pendent from the polymer backbone is the residue of crotonic acid obtained after copolymerization of crotonic acid with vinyl acetate and, optionally, one or more vinyl alkanoates followed by hydrolysis to create alcohol functionality in any desired degree of hydrolysis and the internal lactone formed is a gamma-lactone. In an especially preferred embodiment, the polyvinyl alcohol is derivatized with 1-20 wt % crotonic acid gamma-lactone based on 100 wt % of the polyvinyl alcohol polymer.

In a more preferred embodiment, the polyvinyl alcohol is derivatized with 2-13 wt % crotonic acid gamma-lactone based on 100 wt % of the polyvinyl alcohol polymer.

In an even more preferred embodiment, the polyvinyl alcohol is derivatized with 6-12 wt % crotonic acid gamma-lactone based on 100 wt % of the polyvinyl alcohol polymer.

In a most preferred embodiment, the polyvinyl alcohol is derivatized with 6-12 wt % crotonic acid gamma-lactone based on 100 wt % of the polyvinyl alcohol polymer, which has a degree of hydrolysis of 35-50%.

It should be noted that the carboxylic acid pendent from the polymer that is the residue of acrylic acid or crotonic acid obtained after copolymerization of acrylic acid or crotonic acid, respectively, with vinyl acetate and, optionally, one or more vinyl alkanoates followed by hydrolysis to create alcohol functionality in any desired degree of hydrolysis is known to form a lactone with the alcohol functionality pendent from the polyvinyl alcohol polymer. For this reason, steps are sometimes taken to inhibit the formation of the lactone. However, in a preferred embodiment, steps are not taken to inhibit lactone formation.

In one embodiment, the film-forming polymer is incorporated into the sunscreen formulations to impart waterproofing properties thereto.

In one embodiment, the waterproofing polymer is incorporated into sunscreen formulations in an amount of 0.1 to 10 wt % based on a total weight of the formulation.

In another embodiment, the waterproofing polymer is incorporated into sunscreen formulations in an amount of 0.5 to 5 wt % based on a total weight of the formulation.

In yet another embodiment, the waterproofing polymer is incorporated into sunscreen formulations in an amount of 1 to 3 wt % based on a total weight of the formulation.

The disclosed sunscreen formulations additionally comprise at least one sunscreen active agent. For purposes of the present disclosure, a “sunscreen active agent” is a material, used singly or in combination with other such materials, that is regarded as acceptable for use as an active sunscreening ingredient based on its ability to absorb UV radiation. Such compounds are generally described by their ability to act as UV active agents and their performance in different spectra regions describes as UV-A, UV-B, or UV-A/UV-B. Approval by a regulatory agency is generally required for inclusion of active agents in formulations intended for human use. Those active agents which have been or are currently approved for sunscreen use in the United States include organic and inorganic substances including, without limitation, para aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy triolcate, ethyl dihydroxypropyl PABA, glyceryl aminobenzoate, lawsone with dihydroxyacetone, red petrolatum. Examples of additional sunscreen actives that have not yet been approved in the U.S. but are allowed in formulations sold outside of the U.S. include ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. However, as the list of approved sunscreens is currently expanding, those of ordinary skill will recognize that the disclosure is not limited to sunscreen active agents currently approved for human use but is readily applicable to those that may be allowed in the future.

In Europe, sunscreen active agents approved and, therefore, useful according to the present disclosure, include, again without limitation, benzophenones, for example, Benzophenone-3 (BP3) and Benzophenone-4 (BP4); Salicylates, for example, Homosalate (HMS) and 2-ethylhexyl salicylate (EHS); p-Aminobenzoic acid and derivatives, for example, Ethylhexyl dimethyl PABA (OD-PABA) and 4-p-aminobenzoic acid (PABA); Benzimidazole derivatives, for example, Phenylbenzimidazole sulfonic acid (PMDSA) and Disodium phenyl dibenzimidazole tetrasulfonate (bisdisulizole disodium); Triazines, for example, Ethylhexyltriazone (OT), Diethylhexyl butamido triazone (DBT), and Bis-ethylhexyloxyphenol methoxyphenyl triazine (EMT); Benzotriazoles, for example, Drometrizole trisiloxane (DRT) and Methylene bis-benzotriazolyl tetramethylbutylphenol (MBP, biscotrizole); Dibenzoylmethane derivatives, for example, 4-tert-Butyl-4′-methoxydibenzoylmethane (BM-DBM, avobenzone); Cinnamates, for example, Ethylhexyl methoxycinnamate (OMC) and Isoamyl p-methoxycinnamate (IMC, amiloxate); and Camphor derivatives, for example, Terephtalydene dicamphor sulfonic acid (PDSA), 3-benzylidene camphor (3BC), Benzylidene camphor sulfonic acid (BCSA), 4-methylbenzylidene camphor (4-MBC), Polyacrylamidomethyl benzylidene camphor (PBC), and Camphor benzalkonium methosulfate (CBM).

In one embodiment of the disclosure, the sunscreen active agent comprises a photoprotecting effective amount of particulates of at least one inorganic pigment or nanopigment, non-limiting examples of which include titanium dioxide, zinc oxide, iron oxide, zirconium oxide, cerium oxide, or mixture thereof.

In an especially preferred embodiment, the at least one sunscreen active agent is selected from the group consisting of avobenzene, homosalate, octisalate, octocrylene, and oxybenzone.

Generally, the sunscreen active agent is present in the sunscreen formulation in amounts well-known in the art to be effective to protect a user to be exposed or already exposed to sunlight from the damaging effects of exposure to sunlight. Typically, these amounts range from 1-25% by weight, preferably 3-25% by weight based on a total weight of the sunscreen formulation.

Additionally, the solvent can include an oil such as mineral or vegetable oil in varying amounts as a co-solvent or as described herein as “emollients”.

Emollients can include any appropriate oil, solvent, ester, triglyceride, etc. that is appropriate for the end use application. For suncare products, typical emollients include Triheptanoin, Isopropyl Palmitate, Triheptanoin (and) C13-C16 Isoparaffin, Heptyl Undecylenate, Caprylic/Capric Triglyceride, Diisooctyl Succinate, C13-C16 Isoparaffin (and) Heptyl Undecylenate, C12-C15 alkyl benzoate, Caprylic/Capric Triglyceride, and other appropriate esters.

The sunscreen formulation may additionally comprise other film-forming polymers in addition to the polyvinyl alcohol described herein. Such other film-forming polymers can be selected from, merely for example, starch ester-based polyglucose polymers, such as are described in U.S. Pat. No. 11,135,148, the entire contents of which are hereby incorporated by reference; polyesters, such as are described in US 2021/0259930, US 2021/0259945, and US 2021/0259946, the entire contents of which are hereby incorporated by reference; and N-alkyl (meth)acrylamide copolymers, such as are described in US 20180098930, the entire contents of which are hereby incorporated by reference. Additional examples of suitable other film-forming polymers include film-forming polymer comprising at least 5% by weight, based on a total weight of the film-forming polymer, of an acid-containing monomer. In a preferred embodiment, the film-forming polymer comprises at least 5% by weight of a carboxylic acid-containing monomer. Non-limiting examples of these monomers are acrylic acid, crotonic acid, methacrylic acid, maleic acid, itaconic acid, and combinations and mixtures thereof. Additional film-forming polymers, cither synthetic or natural can be used with the acid-containing polymers described above. Non-limiting examples of these additional film forming polymers are: from Nouryon, AMPHOMER® and AMPHOMER® LV-71 polymers (octylacrylamide/acrylates/butylaminocthyl methacrylate com polymer), AMPHOMER® HC® polymer (acrylates/octylacrylamide copolymer), BALANCE® 0/55 and BALANCE CR® polymers (acrylates copolymer), BALANCE® 47 polymer (octylacrylamide/butylaminocthyl methacrylate copolymer), RESYN® 28-2930 polymer (VA/crotonates/vinyl neodecanoate copolymer), RESYN® 28-1310 polymer (VA/Crotonates copolymer), FLEXAN® polymers (sodium polystyrene sulfonate), DynamX polymer (polyurethane-14 (and) AMP-Acrylates copolymer), RESYN XP® polymer (acrylates/octylacrylamide copolymer), STRUCTURE 2001 (acrylates/steareth-20 itaconate copolymer) and STRUCTURE® 3001 (acrylates/ceteth-20 itaconate copolymer); from ISP, OMNIREZ-2000® (PVM/MA half ethyl ester copolymer), GANEX P-904® (butylated PVP), GANEX V-216® (PVP/hexadecene copolymer) GANEX® V-220 (PVP/cicosene copolymer), GANEX® WP-660 (tricontanyl PVP), GANTREZ® A425 (butyl ester of PVM/MA copolymer), GANTREZ® AN-119 PVM/MA copolymer, GANTREZ ES 225® (ethyl ester of PVM/MA copolymer), GANTREZ ES425 (butyl ester of PVM/MA copolymer), GAFFIX VC-713® (vinyl caprolactam/PVP/dimethylaminocthyl methacrylate copolymer), GAFQUAT755® (polyquaternium-11), GAFQUAT HS-100® (poly-quaternium-28), AQUAFLEX XL-306 (Polyimide-1), AQUAFLEX SF-40® (PVP/Vinylcaprolactam/DMAPA Acrylates Copolymer), AQUAFLEX FX-64® (Isobutylenc/Ethylmalcimide/Hydroxyethylmalcimide Copolymer), ALLIANZ LT-120® (Acrylates/C1-2 Succinates/Hydroxy acrylates Copolymer), STYLEZE CC-10® (PVP/DMAPA Acrylates Copolymer), STYLEZE 2000® (VP/Acrylates/Lauryl Methacrylate Copolymer), STYLEZE W-20® (Poly quaternium-55), Copolymer Series (PVP/Dimethylamino ethylmethacrylate Copolymer), ADVANTAGE S® and ADVANTAGE LCA® (VinylcaprolactamNP/Dimethylaminoethyl Methacrylate Copolymer), ADVANTAGE PLUS (VA/Butyl Maleate/Isobornyl Acrylate Copolymer), Antaron ECo (Ethylcellulose); from BASF, ULTRAHOLD STRONG (acrylic acid/ethyl acrylate/t-butyl acrylamide), LUVIMER 100P® (t-butyl acrylate/ethyl acrylate/methacrylic acid), LUVIMER 36D (ethyl acrylate/t-butyl acrylate/methacrylic acid), LUVIQUAT HM-552® (polyquaternium-16), LUVIQUAT HOLD® (polyquaternium-16), LUVISKOL K30 (PVP) LUVISKOL K90® (PVP), LUVISKOL VA 64® (PVP/VA copolymer), LUVISKOL VA73W® (PVPNA copolymer), LUVISKOL VA®, LUVISET PUR® (Polyurethane-1), LUVISET® Clear (VP/MethacrylamideNinyl Imidazole Copolymer), LUVIFLEX SOFT® (Acrylates Copolymer), ULTRA HOLD 8® (Acrylates/Acrylamide Copolymer), LUVISKOL® Plus (Polyvinylcaprolactam), LUVIFLEX® Silk (PEG/PPG-25/25 Dimethicone/Acrylates Copolymer); from Amerchol, AMERHOLD® DR-25 (acrylic acid/meth acrylic acid/acrylates/methacrylates); from Rohm & Haas, ACUDYNE 258® (acrylic acid/methacrylic acid/acrylates/methacrylates/hydroxy ester acrylates; from Mitsubishi and distributed by Clariant, DIAFORMER Z-301®, DIA FORMER Z-SM®, and DIAFORMER Z-400® (methacryloyl ethyl betaine/acrylates copolymer), ACUDYNE 180® (Acrylates/Hydroxyesters Acrylates Copolymer), ACU DYNE SCP (Ethylenecarboxyamide/AMPSA/Methacrylates Copolymer), and the ACULYN® rheological modifiers; from ONDEO Nalco, FIXOMER A-30® and FIXOMER N-28® (INCI names: methacrylic acid/sodium acrylamidomethyl propane sulfonate copolymer); from Noveon, FIXATE G-100® (AMP-Acrylates/Allyl Meth acrylate Copolymer), FIXATE PLUS® (Polyacrylates-X), CARBOPOL® Ultrez 10 (Carbomer), CARBOPOL Ultrez 20® (Acrylates/C10-30 Alkyl Acrylates Copolymer), AVALLTRE AC® series (Acrylates Copolymer), AVALURE UR® series (Polyurethane-2, Polyurethane-4, PPG-17/IPDI/DMPA Copolymer); polyethylene glycol; water-soluble acrylics; water-soluble polyesters; polyacryl amides; polyamines; polyquaternary amines; styrene maleic anhydride (SMA) resin; polyethylene amine; from Covestro, Baycusan® C 2000 (Polyurethane solution in ethanol (INCI-Polyurethane-64)), Baycusan® cco E 1000 (water-based polyurethane), Baycusan® C 1010 (water-based polyurethane), Baycusan® C 1008 (water-based polyurethane), Baycusan® C 1001 (water-based polyurethane); from Inolex, Inolex Lexfilm Sun (INCI-Polyester-7 (and) Neopentyl Glycol Diheptanoate), Inolex Lexfilm Sun Natural MB (INCI-Capryloyl Glycerin/Sebacic Acid Copolymer), Inolex WetFilm MB (INCI-Trimethylpentanediol/Adipic Acid/Glycerin Crosspolymer), Inolex Lexfilm Spray (INCI-Polyester-10 (and) Propylene Glycol Dibenzoate) and Inolex Lexorez 100 MB (INCI-Adipic Acid/Diglycol Crosspolymer); and other conventional polymers that are polar solvent soluble or that can be made soluble through neutralization with the appropriate base.

In one embodiment, the sunscreen formulation comprises at least one biodegradable starch ester-based polyglucose film-forming polymer in addition to the polyvinyl alcohol described herein.

In another embodiment, the sunscreen formulation comprises at least one biodegradable polyester film-forming polymer in addition to the polyvinyl alcohol described herein.

In another embodiment, the sunscreen formulation comprises at least one biodegradable diisostearoyl polyglyceryl-3 dimer dilinoleate film-forming polymer in addition to the polyvinyl alcohol described herein.

In an especially preferred embodiment, the diisostearoyl polyglyceryl-3 dimer dilinoleate film-forming polymer comprises diisostearoyl polyglyceryl-3 dimer dilinoleate and caprylic/capric triglyceride.

When the sunscreen formulation comprises another film-forming polymer in addition to the polyvinyl alcohol described herein, such other film-forming polymer may also be present in the sunscreen formulation in an amount of 0.1 to 10 wt % based on a total weight of the formulation.

In a preferred embodiment, such other film-forming polymer is incorporated into sunscreen formulations in an amount of 0.5 to 5 wt % based on a total weight of the formulation.

In another preferred embodiment, the combined amounts of all other such film-forming polymers and the polyvinyl alcohol described herein total 0.1 to 10 wt % based on a total weight of the formulation.

In an especially preferred embodiment, the combined amounts of all other such film-forming polymers and the polyvinyl alcohol described herein total 0.5 to 5 wt % based on a total weight of the formulation.

The disclosed sunscreen formulations may contain a wide range of additional, optional components which are referred to herein as “cosmetic components”, but which can also include components generally known as pharmaceutically active agents. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present disclosure. Examples of these functional classes disclosed in this reference include: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), SPF boosters, waterproofing agents, and viscosity increasing agents (aqueous and nonaqueous).

The compositions of this disclosure can be applied to the skin as a liquid rub on, but are most commonly applied as a spray. However, the compositions are not limited to those compositions applied to the skin primarily as a sunscreen agent. The compositions also incorporate those formulations where the sunscreen active agent is an ingredient in another topically applied composition. Some non-limiting examples are lipstick, make-up, lip-balm, eye-shadow, hair dyes and conditioners, or any application where sun protection may be deemed beneficial.

In an embodiment, wherein the disclosed formulation is to be used in an application where sun protection may be deemed beneficial, the disclosed formulation affords a static sun protection factor (“static SPF”). Without being limiting, the SPF could be greater or lesser than 10, or greater than 20, or greater than 25, or greater than 30, or greater than 35, or greater than 40, or greater than 45, or greater than 50, or greater than 55, or greater than 60, or greater than 65, or greater than 70, or greater than 75, or greater than 80, or greater than 85, or even higher.

In an embodiment, wherein the disclosed formulation is to be used in an application where sun protection may be deemed beneficial, the disclosed formulation affords a water resistance sun protection factor (“WR SPF”). Again, without being limiting, the SPF could be greater or lesser than 10, or greater than 20, or greater than 25, or greater than 30, or greater than 35, or greater than 40, or greater than 45, or greater than greater than 50, or greater than 55, or greater than 60, or greater than 65, or greater than 70, or greater than 75, or even higher.

The disclosure will now be described in greater detail with reference to the following non-limiting examples.

### EXAMPLES

### Example 1

### A. Anhydrous Formulation Preparation

Anhydrous formulations were prepared to exemplify a spray product: An anhydrous concentrate was made in a 400 mL beaker, in which 63 grams anhydrous ethanol SDA-40B is charged to the beaker and with mixing, 2 g polyvinyl alcohol derivative or comparative polymer is added and mixed for ˜30 minutes until completely dispersed. With continued mixing, an oil phase including 3 g avobenzone (Neo Heliopan® 357, Symrise), 13 g homosalate (Neo Heliopan® HMS, Symrise), 5 g ethylhexyl salicylate (Neo Heliopan® OS, Symrise), 9 g octocrylene (Neo Heliopan® 303, Symrise), and 5 g C12-15 alkyl benzoate (Finsolv TN, Innospec) is added to the beaker and mixed for ˜30 minutes and the results observed.

TABLE 1

| | Gram | Weight | INCI or | CAS |
|---|---|---|---|---|
| Ingredient | used | Percent | Chemical Name | Number |
| Ethanol Phase | | | | |
| Anhydrous Ethanol | 63.00 | 63.00% | SD Alcohol 40B | 64-17-5 |
| Film-Forming | 2.00 | 2.00% | Polymer of this | |
| Polymer | | | Disclosure or | |
| | | | Comparator | |
| Oil Phase | | | | |
| NEO HELIOPAN ® | 3.00 | 3.00% | Avobenzone | 70356-09-1 |
| 357 | | | | |
| NEO HELIOPAN ® | 13.00 | 13.00% | Homosalate | 118-56-9 |
| HMS | | | | |
| NEO HELIOPAN ® | 5.00 | 5.00% | Octisalate | 118-60-5 |
| OS | | | | |
| NEO HELIOPAN ® | 9.00 | 9.00% | Octocrylene | 6197-30-4 |
| 303 | | | | |
| Finsolv ® TN | 5.00 | 5.00% | C12-15 Alkyl Benzoate | 68411-27-8 |
| Total Weight | 100 | 100.00% | | |

### B. Solubility Results

The desired result is a clear, one-phase, and complete solution, which is taken as an indication that the formulation is fully soluble. On the other hand, if the solution is hazy or contains precipitate, this is taken as an indication that the formulation is at least partly insoluble. Evaluations are made immediately after mixing (“Initial Solubility”) and 24 hours later (“24 Hour Solubility”).

TABLE 2

| | Film-Forming | Degree of | Initial | 24 Hour |
|---|---|---|---|---|
| # | Polymer | Hydrolysis (%) | Solubility | Solubility |
| 1 | Dermacryl ® 79¹ | Not applicable | Clear | Clear |
| 2 | Poval ™ LM-10 HD² | 38-42 | Clear | Clear |
| 3 | Poval ™ LM-20³ | 38-42 | Clear | Clear |
| 4 | Active 35⁴ | 32.7 | Clear | Clear |
| 5 | Active 45⁵ | 44-46 | Clear | Clear |
| 6 | Active 55⁶ | 55.5-57.5 | Clear | Clear |
| 7 | Ethapol XS⁷ | 66.7 | Slight Haze | Precipitate |
| 8 | Other PVOH⁸ | 70-99.9 | Insoluble | Insoluble |

¹Acrylic copolymer available from Nouryon
^{2, 3}Polyvinyl alcohol available from Kuraray
^{4, 5, 6, 7}Polyvinyl alcohol available from Nouryon
⁸We tested (1) Poval ™ 5-74 (DH = 72.5-74.5%); (2) Poval ™ 4-88 (DH = 86.7-88.7); and (3) Poval ™ 4-98 (DH = 98.0-98.8%), and in each case the polymers were completely insoluble in 5 wt %, 2.5 wt %, and 1 wt % anhydrous ethanol.

The results demonstrate a clear correlation between the degree of hydrolysis of the film-forming polymer and the solubility of the formulation in anhydrous ethanol.

Generally speaking, the solubility of any disclosed film-forming polymer in any particular solvent or solvent system can be determined analogously to this example in a simple and straightforward manner.

### Example 2

Derivatives were prepared and tested analogously to Example 1.

The succinates were prepared by reacting the base polyvinyl alcohol polymer with succinic anhydride.

The caprolactone condensation products were prepared by reacting the base polyvinyl alcohol polymer with caprolactone.

The vinyl pivalates were prepared in the manner described in Example 12 below.

The 2-ethylhexanoates were prepared in the manner described in Example 11 below.

The vinyl laurates were prepared in the manner described in Example 10 below.

TABLE 3

| | | Initial | 24 Hour |
|---|---|---|---|
| # | Film-Forming Polymer | Solubility | Solubility |
| 9 | PVA LM-20 Succinate | Clear | Clear |
| | (80:20) | | |
| 10 | PVA LM-20 Succinate | Clear | Clear |
| | (70:30) | | |
| 11 | PVA LM-10HD Succinate | Clear | Clear |
| | (80:20) | | |
| 12 | PVA LM-10HD Succinate | Clear | Clear |
| | (70:30) | | |
| 13 | PVA LM 10HD + Caprolactone | Clear | Clear |
| | (80:20) | | |
| 14 | PVA LM 10HD + Caprolactone | Clear | Clear |
| | (90:10) | | |
| 15 | PVA LM-20 + Caprolactone | Clear | Clear |
| | (90:10) | | |
| 16 | PVA LM-20 + Caprolactone | Clear | Clear |
| | (80:20) | | |
| 17 | PVOH Vinyl Pivalate | Clear | Clear |
| | (93:7; DH 32%) | | |
| 18 | PVOH Vinyl Pivalate | Clear | Clear |
| | (93:7; DH 55%) | | |
| 19 | PVOH Vinyl Pivalate | Clear | Clear |
| | (93:7; DH 73%) | | |
| 20 | PVOH Vinyl Pivalate | Clear | Clear |
| | (93:7; DH 83%) | | |
| 21 | PVOH Vinyl 2-Ethylhexanoate | Clear | Clear |
| | (95:5; DH 19%) | | |
| 22 | PVOH Vinyl 2-Ethylhexanoate | N.D. | N.D. |
| | (95:5; DH 36%) | | |
| 23 | PVOH Vinyl 2-Ethylhexanoate | N.D. | N.D. |
| | (95:5; DH 52%) | | |
| 24 | PVOH Vinyl 2-Ethylhexanoate | Clear | Clear |
| | (95:5; DH 73%) | | |
| 25 | PVOH Vinyl Laurate | Clear | Clear |
| | (96:4; DH 35%) | | |
| 26 | PVOH Vinyl Laurate | Clear | Clear |
| | (96:4; DH 61%) | | |
| 27 | PVOH Vinyl Laurate | Clear | Clear |
| | (96:4; DH 80%) | | |

N.D. = not determined

### Example 3

Selected polymer samples (using the numbering in the tables above) were prepared in ethanol solvent with the oil phase and blended with propellant according to the following procedure to understand compatibility with propellants.

### Formulation Preparation Procedure:

1. Charge all ethanol in the formulation to the main mixing vessel.

2. Begin mixing with propeller agitation (adjust the speed of the speed of the mixing until there is a vortex pulled 2/3 of the way down the mixing shaft).

3. Slowly add the polymer powder by sifting it into the side of the vortex. Allow the polymer to disperse completely.

4. Slowly add the oil phase into the vortex and continue mixing until clear and homogeneous.

5. Fill concentrate into aerosol containers and charge with propellant.

6. Charged aerosols are observed for clarity, phases, and any precipitation that may have formed initially and over time.

In the following examples the propellant is either isobutane at 30% or dimethyl ether (DME) at 30% and 40% of the total formulation. Where the propellant is 30 wt % isobutane, the test formulation comprised 1.4 wt % polymer, ethanol QS to 44.1 wt %, 24.5 wt % UV oil phase, and 30 wt % isobutane propellant. Where the propellant is 30 wt % DME, the test formulation comprised 3 wt % polymer, up to 1 wt % aminomethylpropanol, ethanol QS to 70 wt %, and 30 wt % dimethylether propellant. Where the propellant is 40 wt % DME, the test formulation comprised 3 wt % polymer, up to 1 wt % aminomethylpropanol, ethanol QS to 60 wt %, and 40 wt % dimethylether propellant.

The desired result is a clear, one-phase, and complete solution, which is taken as an indication that the formulation is compatible. On the other hand, if the solution is hazy or opaque or contains precipitate, this is taken as an indication that the formulation is at least partly incompatible.

TABLE 4

| | | 30% | 30% | 40% |
|---|---|---|---|---|
| # | Film-Forming Polymer | Isobutane | DME¹ | DME |
| 1 | Dermacryl ® 79 | Clear | Clear | Clear |
| 2 | Poval ™ LM-10 HD | Incompatible | Clear | Clear |
| 3 | Poval ™ LM-20 | Incompatible | Clear | Clear |
| 4 | Active 35 | Precipitate | Incompatible | Incompatible |
| 5 | Active 45 | Precipitate | Borderline | Borderline |
| 6 | Active 55 | Precipitate | Incompatible | Incompatible |
| 7 | Ethapol XS | Precipitate | Incompatible | Incompatible |
| 9 | PVA LM-20 Succinate | Incompatible | Clear | Clear |
| | (80:20) | | | |
| 10 | PVA LM-20 Succinate | Incompatible | Clear | Clear |
| | (70:30) | | | |
| 11 | PVA LM-10HD Succinate | Incompatible | Clear | Clear |
| | (80:20) | | | |
| 12 | PVA LM-10HD Succinate | Incompatible | N.D. | Incompatible |
| | (70:30) | | | |
| 13 | PVA LM 10HD + Caprolactone | Incompatible | Clear | Clear |
| | (80:20) | | | |
| 14 | PVA LM 10HD + Caprolactone | Incompatible | Clear | Clear |
| | (90:10) | | | |
| 15 | PVA LM-20 + Caprolactone | Incompatible | Clear | Clear |
| | (90:10) | | | |
| 16 | PVA LM-20 + Caprolactone | Incompatible | Clear | Clear |
| | (80:20) | | | |
| 17 | PVOH Vinyl Pivalate | N.D. | Clear | Clear |
| | (93:7; DH 32%) | | | |
| 18 | PVOH Vinyl Pivalate | N.D. | Clear | Clear |
| | (93:7; DH 55%) | | | |
| 19 | PVOH Vinyl Pivalate | N.D. | Clear | Clear |
| | (93:7; DH 73%) | | | |
| 20 | PVOH Vinyl Pivalate | N.D. | Clear | Clear |
| | (93:7; DH 83%) | | | |
| 21 | PVOH Vinyl 2-Ethylhexanoate | N.D. | Clear | Clear |
| | (95:5; DH 19%) | | | |
| 22 | PVOH Vinyl 2-Ethylhexanoate | N.D. | Clear | Clear |
| | (95:5; DH 36%) | | | |
| 23 | PVOH Vinyl 2-Ethylhexanoate | N.D. | Clear | Clear |
| | (95:5; DH 52%) | | | |
| 24 | PVOH Vinyl 2-Ethylhexanoate | N.D. | Clear | Clear |
| | (95:5; DH 73%) | | | |
| 25 | PVOH Vinyl Laurate | N.D. | Clear | Clear |
| | (96:4; DH 35%) | | | |
| 26 | PVOH Vinyl Laurate | N.D. | Clear | Clear |
| | (96:4; DH 61%) | | | |
| 27 | PVOH Vinyl Laurate | N.D. | Clear | Clear |
| | (96:4; DH 80%) | | | |

¹dimethyl ether (DME)
N.D. = not determined

The results show that solubility of the base polymer in anhydrous ethanol does not guarantee compatibility with propellants. None of the disclosed film-forming polymers proved to be compatible with isobutane propellant. However, a number of the disclosed film-forming polymers were compatible with 30% and 40% DME.

Generally speaking, the compatibility of any disclosed film-forming polymer and a particular propellant or solvent-propellant system can be determined analogously to this example in a simple and straightforward manner.

### Example 4

In order to demonstrate the effectiveness of the sunscreen formulations examples made with polyvinyl alcohols of this disclosure, or lack of effectiveness of formulations made with comparative products, for each formulation made, a series of tests are carried out to quantify the so-called Sun Protection Factor (SPF) and to demonstrate their effectiveness in creating a demonstrable water-resistant suncare formulation.

Due to the importance of SPF testing for labeling consumer suncare products, governmental agencies regulate the labeling of these products and require testing of the products on human panelists with specific statistical criteria. (Static) SPF measures the ratio of the time it takes for sunscreen protected skin to show the onset of erythema as compared to a non-sunscreen protected area of skin on the same human panelist.

Water-resistant (WR) SPF measurements likewise do that after a standard immersion of the skin in water at 40° C.

For laboratory measurements of SPF and WR SPF, a series of In-Vitro measurements can be made. Colipa Test methods are used in Europe. FDA 21 CFR Section 201 and 320 tests are used in the US. These measurements involved using plastic films or substrates to simulate the roughness and other properties of human skin. In this case, the substrate used SPF measurements can be taken using a variety of instruments. We used a Labsphere UV-1000 Spectrophotometer. Artificial skins including partially-hydrophilic acrylic polymer material, Vitro Skin™ from Florida Suncare, Inc., (formerly IMS Inc) were used following manufacturer instructions.

The film was coated with 20×5 mg drops of sunscreen concentrate, which were carefully and evenly distributed over the 55.8 cm2 area of the film, again coating with 2 mg/cm2. The UV spectrophotometer was used to measure the absorption (or transmission) of both UVA radiation (320-400 nm) and UVB radiation (290-320 nm). From these values the In-Vitro SPF could be measured for the skin with and without suncare product applied to the artificial skin.

The results are reported as In-Vitro SPF and In-Vitro WR SPF, where the former is again the measurement after a standard drying time and the latter is the measurement of the same film after 80 minutes immersion in water held at 40° C. These In-Vitro measurements have been shown to correlate to In-Vivo data. Other data such as UVA and UVB ratio can also be determined and used to quantify protection provided by the sunscreen formulations. Alternatively, for the Colipa Tech method, Helioplate HD6 embossed PMMA plates are available from Helio Labs Inc. These PPMA plates have a 6 micrometer root mean square surface roughness and are used without hydration. Due to the depth of the roughness, the same 2 mg/cm2 dose of sunscreen emulsion, once coated and dried on the plate, will have “peaks” of the surface roughness which are covered with relatively less UV absorbing oil. Thus, the absorbance and calculated SPF can be a bit reduced relative to in-vivo and other in-vitro measurement techniques. However, both in-vitro substrates give reproducible results and can be compared using appropriate correction factors.

Thus, as is standard in the art, UV absorption through plastic films coated with UV-absorbing suncare formulation is used to mimic human skin and is measured with a spectrophotometer to provide a measure of sun protection factor (SPF). SPF is simply the ratio of the initial light to the transmitted light through the UV absorbing film. If 100% is reduced to 10%, the SPF is 10. If 100% is reduced to 1%, the SPF is 100. SPF 50 corresponds to 2% of the UV light being transmitted through the skin.

In each case, a UV-absorbing film having a surface area of 55.8 cm2 was coated with 20×5 mg drops of a solution containing the test polymer and subjected to UV light in the 280 to 400 nm range to provide a so-called “static” SPF measurement.

In-Vitro Water-Resistance SPF (WR SPF) measurements are taken from the same film after it is placed in a water-bath, heated to 40° C., and subjected to mild agitation. The sample after being withdrawn is gently dried under reproducible conditions and again measured for light transmission. this time providing WR SPF data.

TABLE 5

| | | Static | WR |
|---|---|---|---|
| # | Film-Forming Polymer | SPF | SPF |
| 1 | Dermacryl ® 79 | 56.6 | 55.8 |
| 2 | Poval ™ LM-10 HD | 62.7 | 51 |
| 3 | Poval ™ LM-20 | 51.2 | 33.3 |
| 4 | Active 35 | 56.2 | 33.6 |
| 5 | Active 45 | 47.6 | 39.3 |
| 6 | Active 55 | 63.1 | 65.1 |
| 7 | Ethapol XS | 71.4 | 74.5 |
| 9 | PVA LM-20 Succinate | 72.5 | 59.9 |
| | (80:20) | | |
| 10 | PVA LM-20 Succinate | 73.9 | 55.9 |
| | (70:30) | | |
| 11 | PVA LM-10HD Succinate | 69.8 | 47.6 |
| | (80:20) | | |
| 12 | PVA LM-10HD Succinate | 83.8 | 50.3 |
| | (70:30) | | |
| 13 | PVA LM 10HD + Caprolactone | 33.6 | 37.8 |
| | (80:20) | | |
| 14 | PVA LM 10HD + Caprolactone | 25.3 | 32.5 |
| | (90:10) | | |
| 15 | PVA LM-20 + Caprolactone | 37.4 | 41.4 |
| | (90:10) | | |
| 16 | PVA LM-20 + Caprolactone | 34.5 | 34.2 |
| | (80:20) | | |
| 17 | PVOH Vinyl Pivalate | 52.2 | 46.1 |
| | (93:7; DH 32%) | | |
| 18 | PVOH Vinyl Pivalate | 50.8 | 39.9 |
| | (93:7; DH 55%) | | |
| 19 | PVOH Vinyl Pivalate | 63.0 | 65.0 |
| | (93:7; DH 73%) | | |
| 20 | PVOH Vinyl Pivalate | 55.7 | 54.0 |
| | (93:7; DH 83%) | | |
| 21 | PVOH Vinyl 2-Ethylhexanoate | 37.2 | 28.9 |
| | (95:5; DH 19%) | | |
| 24 | PVOH Vinyl 2-Ethylhexanoate | 25.4 | 9.7 |
| | (95:5; DH 73%) | | |
| 25 | PVOH Vinyl Laurate | 53.6 | 49.7 |
| | (96:4; DH 35%) | | |
| 26 | PVOH Vinyl Laurate | 48.4 | 3.0 |
| | (96:4; DH 61%) | | |
| 27 | PVOH Vinyl Laurate | 65.6 | 2.6 |
| | (96:4; DH 80%) | | |

### Example 5

Pair Comparison-blind evaluations are performed by 8 panelists using two different leave-on products by applying them to their volar forearm. The panelist has to select one product as applied to their volar forearm as more intense than the other in each performance attribute. Performance data is summarized and analysed statistically.

### Skin Preparation:

Panelists will clean the entire volar forearm area as well as their evaluation fingers using provided ethanol wipes. Once the ethanol has fully evaporated an approximately 1.50″ diameter circle will be drawn on the forearm as the evaluation site and labelled alphabetically (A or B). Use caution to avoid the wrist and crook of the arm areas and provide sufficient space between samples as to ensure they don't run into one another during application.

### Application Evaluation by Panelists:

1. Provide the panelists with the evaluation ballot for recording their answers

2. Making sure the panelists are unaware of the identification of the sample, an electronic pipette should be used to apply approximately 0.4 μL of each sample (A and B) to the center of the evaluation sites.

3. The panelists spread the measured amount of product within the circle using the index or middle finger, using a gentle circular motion-stroking at a rate of approximately 2 strokes per second.

4. Indicated below are the attributes that panelists evaluated for comparison of the experimental sunscreen solution vs. a control sunscreen solution (DERMACRYL® 79; Acrylates/Octylacrylamide Copolymer, Nouryon) in the sunscreen formulation as outlined above:

### Spreadability: Ease of Moving Product Over the Skin (Difficult/Drag to Easy/Slip)

After 10 rubs, evaluate and choose which product has the highest intensity of the following:

Stickiness: Degree to which fingers adhere to residual product (not sticky to very sticky)

Visually analyse the forearm test site for evaluate for:

Gloss: Amount or degree of light reflected off skin (none to high amount)

Immediate After-feel: Stroke cleansed fingers (1-2 strokes) lightly across skin and evaluate for:

Slipperiness: Ease of moving fingers over the skin (difficult/drag to easy/slip)

Amount of Residue: Amount of product on skin (none to large amount)

Oiliness: A slippery, smooth, continuous feel (i.e. baby oil)

Powderiness: A thin, slippery coating that is very dry (i.e. corn starch)

Assess stickiness again, after product has had time to absorb into the skin after ˜5 minutes:

Stickiness2: Degree to which fingers adhere to residual product (not sticky to very sticky)

At the end of the attributes assessment, panelists are asked to select which product was preferred, indicated by “overall preference”.

### Analysis of Data/Assessments:

Sample exhibits less of the attribute: experimental selected 0 to 1 times out of 8 showing a statistically significant difference from the reference at a 95% confidence level

Sample show no statistical difference to standard: experimental sample selected 2 to 6 times out of 8

Sample exhibits more of the property: experimental selected 7 to 8 times out of 8 showing a statistically significant difference from the reference at a 95% confidence level

For spreadability, slipperiness, and preference it is desirable to have more of the property; for stickiness, gloss, residue, and oily it is desirable to have less of the property.

TABLE 6

| | Film-Forming Polymer | | |
|---|---|---|---|
| | Sensory Properties Compared to Dermacryl ® 79 | | |
| | Poval ™ | | PVA LM-20 |
| Property↓ | LM-10 HD | Active 55 | Succinate (80:20) |
| Spreadability | 4 | 5 | 5 |
| Stickiness | 1 | 2 | 2 |
| Gloss | 3 | 4 | 4 |
| Slipperiness | 4 | 5 | 8 |
| Residue | 3 | 7 | 6 |
| Oily | 3 | 4 | 5 |
| Powdery | 4 | 4 | 3 |
| Stickiness 2 | 3 | 3 | 7 |
| Preference | 4 | 5 | 3 |

### Example 6

Several examples of this disclosure were tested by OECD biodegradability method 301D. details of which are readily available. Testing was performed in both river water and activated sludge.

TABLE 7

Film-Forming Polymer
Biodegradability in River Water

| Biodegradation | Poval ™ | |
|---|---|---|
| percentage at Day X↓ | LM-10 HD | Active 55 |
| 7 | 1 | 3 |
| 14 | 7 | 6 |
| 21 | 13 | 17 |
| 28 | 18 | 24 |
| 42 | 35 | 37 |
| 56 | 55 | 52 |
| 84 | 61 | 61 |

TABLE 8

Film-Forming Polymer
Biodegradability in Activated Sludge

| Biodegradation | Poval ™ | |
|---|---|---|
| percentage at Day X↓ | LM-10 HD | Active 55 |
| 7 | 1 | 5 |
| 14 | 1 | 10 |
| 21 | 12 | 15 |
| 28 | 21 | 24 |
| 42 | 38 | 42 |
| 56 | 43 | 57 |
| 84 | 44 | 62 |

The OECD 301D ratings include (1) “Ultimately Biodegradable,” meaning ≥60% biodegradable within a 60 day period; (2) “Readily Biodegradable,” meaning >60% biodegradation in 28 days or less; (3) “Inherently Biodegradable,” meaning 20% to 60% biodegradation in 28 days; and (4) “Non-Biodegradable,” meaning less than 20% biodegradation in 28 days.

### Example 7: Preparation of Intermediate Copolymer with Pendent Acetate Functionality; Precursor to PVOH Copolymers Esterified with Lauric Acid

A four-neck 1 L round bottom flask was equipped with a mechanical stirrer; a Claisen adapter fitted with a temperature probe and a reflux condenser; a 500 mL addition funnel (not pressure equalized); and a 125 mL addition funnel (not pressure equalized). To the flask was charged 7.50 g vinyl laurate, 67.5 g vinyl acetate, 51.15 g ethyl acetate, and 2.91 g 98% tert-butyl peroctoate. To the 500 mL addition funnel was charged a mixture of 22.50 g vinyl laurate and 202.50 g vinyl acetate (Monomer SA-1). To the 125 mL addition funnel was charged a mixture of 2.91 g 98% tert-butyl peroctoate and 67.95 g ethyl acetate (Initiator SA-2).

The reaction mixture was brought to reflux with stirring using a hot water bath. After the reaction mixture was at reflux for 5 minutes, the uniform addition of the contents of Monomer SA-1 to the reaction mixture over 4 h. was started. When Monomer SA-1 addition was complete, the addition funnel was flushed (into the reactor) with 4.61 g ethyl acetate. After the reaction mixture was at reflux for 1h., the uniform addition of the contents of Initiator SA-2 to the reaction over 5 h. was started.

After the addition of Initiator SA-2 was complete, the reaction was held at reflux for an additional 2 h. (total reaction time to this point: 8 h. The reaction was then allowed to cool below reflux, and 70.0 g ethanol was added. After a uniform solution was obtained, the reaction was allowed to stand overnight.

After standing overnight, the reaction was brought back to reflux, and a solution of 0.90 g tert-butyl peroctoate in 7.50 g ethanol was added in a single shot. Heating at reflux was continued for 2 h. after the shot-wise addition of the initiator. The reaction was then allowed to cool below reflux, and 104.0 g ethanol was added. The reaction was stirred until a uniform polymer solution was obtained, and then it was allowed to cool. A clear, colorless viscous polymer solution was obtained. Solids (determined gravimetrically): 49.94%.

### Examples 8 and 9: Preparation of Intermediate Copolymers with Pendent Acetate Functionality; Precursors to PVOH Copolymers Esterified with 2-Ethylhexanoic Acid and Pivalic Acid, Respectively

Additional intermediate copolymers were prepared following the general procedure described in Example 7 using different hydrophobic esters of vinyl alcohol. The compositions of all intermediate copolymers as estimated by the feed ratio of the two monomers are summarized in Table 9 below.

TABLE 9

| | Monomer 1 | Monomer 2 |
|---|---|---|
| Intermediate | (wt. %) | (wt. %) |
| Example 7 | Vinyl acetate | Vinyl laurate |
| | (90) | (10) |
| Example 8 | Vinyl acetate | Vinyl 2- |
| | (90) | ethylhexanoate |
| | | (10) |
| Example 9 | Vinyl acetate | Vinyl pivalate |
| | (90) | (10) |

### Example 10: Partial Hydrolysis of Example 7 Intermediate Copolymer to Give PVOH Polymers Esterified with Lauric Acid

A four-neck 1L round bottom flask was equipped with a mechanical stirrer; a Claisen adapter fitted with a temperature probe and a reflux condenser-topped Barrett trap; a septum; and a stopper. To the flask was charged 250.5 g of the polymer solution prepared in Example 7 (125.0 g, solids basis) and 146 g ethanol. The resulting mixture was stirred until a homogeneous solution was obtained and then 2.25 g of 98% sulfuric acid was added to the reaction mixture. The reaction was brought to a vigorous reflux using a hot water bath, and a total of 40 g of ethanol was distilled from the reaction via the Barrett trap. Heating was continued at mild reflux (no more distillation of ethanol) for 7 h. At 2 h. and 4.5 h. reaction time, about 65 g of the reaction mixture was withdrawn, stirred with about 4.5 g Amberlyst A21 weak basic ion exchange resin for about 1 h., filtered to remove the ion exchange resin, then pan-dried to a constant weight in a 60° C. forced air oven. A sample of the reaction mixture at the end of the reaction (at 7 h.) was similarly processed. In this manner, three partially hydrolyzed PVOH polymers esterified with lauric acid that differed in the degree of hydrolysis were obtained. The compositions are summarized in Table 10.

### Examples 11 and 12: Partial Hydrolysis of Example 8 and 9 Intermediate Copolymers to Give PVOH Polymers Esterified with 2-Ethylhexanoic Acid and Pivalic Acid, Respectively

The precursor polymers from Examples 8 and 9 were partially hydrolyzed following the same general procedure described in Example 10. The compositions arc summarized in Table 10 below.

TABLE 10
Hydrophobic acid used to esterify partially hydrolyzed PVOH: Lauric acid

| | Monomer | | | | | | | |
|---|---|---|---|---|---|---|---|---|
| | Hydrolysis | Vinyl | | Vinyl | | Vinyl | | |
| Film-Forming | Reaction | laurate | | Acetate | | Alcohol | | DH |
| Polymer #↓ | Time | wt. % | mole % | wt. % | mole % | wt. % | mole % | (%) |
| 25 | 2 h | 11 | 4 | 75 | 71 | 14 | 25 | 26 |
| 26 | 4.5 h | 13 | 4 | 59 | 50 | 28 | 46 | 48 |
| 27* | 7 h | 14 | 4 | 43 | 33 | 42 | 63 | 66 |

Hydrophobic acid used to esterify partially hydrolyzed PVOH: 2-Ethylhexanoic acid

| | Monomer | | | | | | | |
|---|---|---|---|---|---|---|---|---|
| | Hydrolysis | Vinyl 2- | | Vinyl | | Vinyl | | |
| Film-Forming | Reaction | ethylhexanoate | | Acetate | | Alcohol | | DH |
| Polymer # ↓ | Time | wt. % | mole % | wt. % | mole % | wt. % | mole % | (%) |
| 21 | 1.5 h | 11 | 5 | 80 | 78 | 9 | 17 | 18 |
| 22 | 3 h | 12 | 5 | 72 | 66 | 16 | 29 | 31 |
| 23 | 5 h | 13 | 5 | 60 | 50 | 28 | 45 | 47 |
| 24 | 7 h | 14 | 5 | 43 | 32 | 43 | 63 | 66 |

Hydrophobic acid used to esterify partially hydrolyzed PVOH: Pivalic acid

| | Monomer | | | | | | | |
|---|---|---|---|---|---|---|---|---|
| | Hydrolysis | Vinyl | | Vinyl | | Vinyl | | |
| Film-Forming | Reaction | Pivalate | | Acetate | | Alcohol | | DH |
| Polymer # ↓ | Time | wt. % | mole % | wt. % | mole % | wt. % | mole % | (%) |
| 17 | 2 h | 11 | 7 | 76 | 70 | 13 | 23 | 25 |
| 18 | 4 h | 12 | 7 | 63 | 53 | 24 | 40 | 43 |
| 19 | 6 h | 14 | 7 | 47 | 35 | 40 | 58 | 62 |
| 20* | 8 h | 15 | 7 | 36 | 25 | 50 | 68 | 73 |

*Composition determined by Carbon-13 NMR (DMSO-d₆ solvent)

### Example 13: Polyvinyl Alcohol-Crotonic Acid Copolymers

A series of intermediate copolymers with carboxylic acid pendent from the backbone-precursors to PVOH copolymers modified with an internal gamma-lactone (cyclic ester)-were prepared by copolymerizing vinyl acetate with crotonic acid in varying relative amounts following the general procedure described in Example 7. The compositions of these intermediate copolymers as determined by Carbon-13 NMR (DMSO-d6 solvent) are summarized in Table 11 below.

TABLE 11

| | Monomer | |
|---|---|---|
| Intermediate | Vinyl Acetate | Crotonic Acid |

| copolymer #↓ | wt. % | mole % | wt. % | mole % |
|---|---|---|---|---|
| 34 | 92 | 92 | 8 | 8 |
| 35 | 94 | 94 | 6 | 6 |
| 36 | 96 | 96 | 4 | 4 |
| 37 | 98 | 98 | 2 | 2 |

The intermediate (precursor) copolymers were partially hydrolyzed following the same general procedure described in Example 10. The compositions as determined by Carbon-13 NMR (DMSO-d6 solvent) are summarized in Table 12 below. It should be noted that the majority of the carboxylic acid moieties pendent from the intermediate (precursor) copolymers spontaneously formed (cylic) esters (internal gamma-lactones) with alcohols pendent from the nascent PVOH copolymer under the conditions employed to partially hydrolyze the intermediate (precursor) copolymers; in some cases, a small fraction (<40%) of the carboxylic acid moieties pendent from the intermediate (precursor) copolymers did not form (cyclic) esters with alcohols pendent from the nascent PVOH copolymer.

TABLE 12

| | Monomer | | | | | | | | | |
|---|---|---|---|---|---|---|---|---|---|---|
| | Vinyl | | Vinyl | | Crotonic Acid/ | | Crotonic | | | |
| | Acetate | | Alcohol | | γ-Lactone | | acid | | DH | Tg |
| Copolymer #↓ | wt. % | mole % | wt. % | mole % | wt. % | mole % | wt. % | mole % | (%) | (° C.) |
| 28^{a} | 56 | 43 | 32 | 48 | 10 | 6 | 3 | 2 | 53 | 64.4 |
| 29^{a} | 59 | 48 | 27 | 43 | 11 | 7 | 2 | 2 | 47 | 62.8 |
| 30^{b} | 69 | 58 | 22 | 36 | 8 | 5 | 1 | 2 | 38 | 57.2 |
| 31^{c} | 67 | 54 | 27 | 42 | 5 | 3 | 1 | 1 | 44 | 53.2 |
| 32^{d} | 54 | 39 | 42 | 59 | 4 | 2 | 0 | 0 | 60 | 49.4 |
| 33^{d} | 61 | 46 | 35 | 52 | 4 | 2 | 0 | 0 | 53 | 50.8 |

^{a}Intermediate (precursor) polymer: 34.
^{b}Intermediate (precursor) polymer: 35.
^{c}Intermediate (precursor) polymer: 36
^{d}Intermediate (precursor) polymer: 37.

### Example 14

Selected polymer samples having the composition in the table below were prepared in ethanol solvent and blended with propellant according to the procedure of Example 3 to understand compatibility with propellants.

In the following examples the propellant is HPC 152A at 40% of the total formulation. In each case, the test formulation comprised 3% polymer, 5% water, q.s. ethanol, and 40% hydrofluorocarbon 152A propellant.

The desired result is a clear, one-phase, and complete solution, which is taken as an indication that the formulation is compatible. On the other hand, if the solution is hazy or opaque or contains precipitate, this is taken as an indication that the formulation is at least partly incompatible.

TABLE 13

| | Monomer | | | | | | | | | |
|---|---|---|---|---|---|---|---|---|---|---|
| | Vinyl | | Vinyl | | Crotonic Acid/ | | Crotonic | | Vinyl | |
| | Acetate | | Alcohol | | γ-Lactone | | Acid | | Pivalate | |
| Copolymer #↓ | wt. % | mole % | wt. % | mole % | wt. % | mole % | wt % | mole % | wt. % | mole % |
| 38 | 59.18 | 48.00 | 27.11 | 43.00 | 11.24 | 7.00 | 2.47 | 2.00 | | |
| 39 | 44.89 | 38.00 | 26.58 | 44.00 | 13.85 | 9.00 | 2.36 | 2.00 | 12.31 | 7.00 |
| 40 | 39.16 | 32.00 | 31.92 | 51.00 | 14.35 | 9.00 | | | 14.58 | 8.00 |

In all cases, the copolymers proved to be compatible with the formulation, resulting in one-phase aerosols that were very slightly hazy.

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A sunscreen formulation comprising:
(a) a volatile solvent;
(b) at least one film-forming polymer comprising at least one of: (i) polyvinyl alcohol or (ii) polyvinyl alcohol ester, said polyvinyl alcohol ester having ester functionality other than solely acetate, and (i) and (ii) being soluble in the volatile solvent; and
(c) at least one sunscreen active agent.

2. The sunscreen formulation according to claim 1, wherein the volatile solvent is an alcohol-based solvent system, wherein the alcohol-based solvent system comprises at least one C₁₋₆ straight or branched chain alcohol.

3. The sunscreen formulation according to claim 2, wherein the alcohol-based solvent system comprises ethanol.

4. The sunscreen formulation according to claim 1, which comprises 0-20%% by weight of water.

5. The sunscreen formulation according to claim 1, which does not comprise water.

6. The sunscreen formulation according to claim 5, which comprises ethanol but not any other alcohol.

7. The sunscreen formulation according to claim 1, wherein the at least one sunscreen active agent is chosen from para aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, bis-ethylhexyloxyphenol methoxyphenyltriazine, 4-methylbenzylidenecamphor, homosalate, butyl methoxydibenzoylmethane, octocrylene, octyl salicyclate, bemotrizinol and isopentyl 4-methoxycinnamate, benzophenone-3 (BP3), benzophenone-4 (BP4), homosalate (HMS), 2-ethylhexyl salicylate (EHS), ethylhexyl dimethyl PABA (OD-PABA), 4-p-aminobenzoic acid (PABA), phenylbenzimidazole sulfonic acid (PMDSA), disodium phenyl dibenzimidazole tetrasulfonate (bisdisulizole disodium), ethylhexyltriazone (OT), diethylhexyl butamido triazone (DBT), bis-ethylhexyloxyphenol methoxyphenyl triazine (EMT), drometrizole trisiloxane (DRT), methylene bis-benzotriazolyl tetramethylbutylphenol (MBP, biscotrizole), 4-tert-butyl-4′-methoxydibenzoylmethane (BM-DBM, avobenzone), ethylhexyl methoxycinnamate (OMC), isoamyl p-methoxycinnamate (IMC, amiloxate), terephtalydene dicamphor sulfonic acid (PDSA), 3-benzylidene camphor (3BC), benzylidene camphor sulfonic acid (BCSA), 4-methylbenzylidene camphor (4-MBC), polyacrylamidomethyl benzylidene camphor (PBC), camphor benzalkonium methosulfate (CBM), titanium dioxide, zinc oxide, iron oxide, zirconium oxide, cerium oxide, and mixtures thereof.

8. The sunscreen formulation according to claim 7, wherein the at least one sunscreen active agent is chosen from avobenzene, homosalate, octisalate, octocrylene, oxybenzone, and combinations thereof.

9. The sunscreen formulation according to claim 1, wherein the at least one film-forming polymer comprises polyvinyl alcohol having a degree of hydrolysis having a degree of hydrolysis of about 35% to about 58%.

10. The sunscreen formulation according to claim 1, wherein the at least one film-forming polymer comprises an ester of polyvinyl alcohol.

11. The sunscreen formulation according to claim 1, which is a sprayable sunscreen formulation.

12. The sunscreen formulation according to claim 11, which further comprises one or more additives chosen from film-forming polymers, plasticizers, UV absorbers, dyes, perfumes, preservatives, viscosity modifiers, vitamins, moisturizers, anti-itch ingredients, and combinations thereof.

13. The sunscreen formulation according to claim 1, which is an aerosol formulation, and further comprises a propellant.

14. The sunscreen formulation according to claim 13, wherein the propellant is dimethyl ether (DME).

15. The sunscreen formulation according to claim 14, wherein the propellant is 1,1-difluoroethane.

16. The sunscreen formulation according to claim 13, which comprises a bag-on-valve device comprising an insert comprising the sunscreen formulation and propellant surrounding the insert.

17. A method of protecting a user to be exposed or already exposed to sunlight from the damaging effects of exposure to sunlight, said method comprising applying to skin of said user an effective amount therefor of a sunscreen formulation according to claim 1.
